(19) United States
(12) Patent Application Publication
(10) Pub. No.: US 2018/0015122 A1
(43) Pub. Date: Jan. 18, 2018

(54) PHARMACEUTICAL COMPOSITION EFFECTIVE IN PREVENTING ADVERSE EFFECTS ASSOCIATED WITH THE USE OF GLUCOCORTICOIDS

(71) Applicant: Companion Supplements, LLC, Sunrise, IL (US)

(72) Inventors: Julio César Villamil Torres, Bogota (CO); Camilo Rey Ferro, Sunrise, FL (US)

(21) Appl. No.: 15/210,438

(22) Filed: Jul. 14, 2016

(51) Int. Cl. A61K 33/06; A61K 31/593; A61K 31/555; A61K 31/198; A61K 31/197; A61K 31/122; A61K 9/48; A61K 33/00; A61K 9/00

(52) U.S. Cl. CPC A61K 33/06; A61K 33/00; A61K 31/593; A61K 31/555; A61K 9/0053; A61K 31/197; A61K 31/122; A61K 9/4825; A61K 31/198

(57) ABSTRACT

The present invention relates to pharmaceutical compositions comprising combinations of amino acids, vitamins, and minerals, which prevent the occurrence of adverse effects associated with prolonged use of glucocorticoids (GCs), especially those of particular relevance due to their high frequency and potential debilitating effect for the patient. These effects are steroid myopathy, hyperglycemia and loss of bone mass.

### FIELD OF THE INVENTION

The present invention relates to pharmaceutical compositions comprising combinations of amino acids, vitamins, and minerals, useful in preventing adverse effects associated with extended use of glucocorticoids (GCs).

### BACKGROUND OF THE INVENTION

Glucocorticoids (GCs) are steroidal-type chemical compounds with potent suppressive activity of inflammation. GCs have the ability to prevent or suppress inflammation in response to several inciting events, such as radiant, mechanical, chemical, infectious, and immune stimuli.

Although the use of GCs as anti-inflammatory drugs does not address the root cause of the disease, the suppression of inflammation has an enormous clinical utility, which has lead that these products are among the most frequently prescribed.

In this sense, GCs are very useful for treating diseases originating from undesirable immune reactions, including illnesses that occur predominantly by the mechanism of humoral immunity, such as urticaria.

According to their origin, GCs could be divided into two groups: natural and synthetic GCs. Cortisol is the main naturally occurring glucocorticoid, which is synthesized by suprarenal glands, and works by regulating inflammation and other processes in the body.

On the other hand, the synthetic GCs are drugs acting in a similar way to cortisol, which can become even more potent than the naturally occurring GCs.

Below is a list of the GCs most frequently used in drug therapy:

The GCs are currently among the most widely used drugs in many diseases due to their ability to exert effects on almost all systems, thanks partly to the diversity of their actions.

In that way, the therapeutic applications of GCs can be divided into five main groups: autoimmune diseases, allergies, adrenal insufficiency, different types of cancer, and surgery.

Below is a detailed list of the different therapeutic applications of GCs:

However, therapies involving prolonged use of GCs give rise to the development of serious adverse effects, which means that these drugs should be used with extreme caution and careful consideration in relation to the risks and benefits for each patient, generating some fear regarding their use.

Indeed, the adverse effects due to long-term use of high doses of GCs are common and potentially serious.

Among the adverse effects from extended use of GCs, three of them are the most significant, due to their high frequency and potential debilitating effect for the patient, namely steroid myopathy, hyperglycemia, and loss of bone mass.

The truthfulness and severity of the adverse effects related to GCs have been thoroughly verified, to the point that in the document “Highlights of Prescribing Information” for prednisone, methylprednisolone, and in general for all GCs marketed in the United States, a warning about them appears in the following terms:

Steroid Myopathy:

“An acute myopathy has been observed with the use of high doses of corticosteroids, most often occurring in patients with neuromuscular transmission disorders (e.g., myasthenia gravis), or in patients receiving concomitant therapy with neuromuscular blocking drugs (e.g., pancuronium). This acute myopathy is generalized, may involve ocular and respiratory muscles, and may result in quadriparesis. Elevation of creatine kinase may occur. Clinical improvement or recovery after stopping corticosteroids may require weeks to years.”

Hyperglycemia

“Hypothalamic-pituitary-adrenal (HPA) axis suppression, Cushing's syndrome, and hyperglycemia: monitor patients for these conditions with chronic use. Taper doses gradually for withdrawal after chronic use.”

Decreasing Bone Density

“Corticosteroids decrease bone formation and increase bone resorption both through their effect on calcium regulation (i.e., decreasing absorption and increasing excretion) and inhibition of osteoblast function. This, together with a decrease in the protein matrix of the bone secondary to an increase in protein catabolism, and reduced sex hormone production, may lead to inhibition of bone growth in children and adolescents and the development of osteoporosis at any age. Special consideration should be given to patients at increased risk of osteoporosis (i.e., postmenopausal women) before initiating corticosteroid therapy, and bone density should be monitored in patients on long-term corticosteroid therapy.”

It was found that the three adverse effects referred above, which usually occur simultaneously in patients taking GCs chronically and in high doses, produce a common consequence, with debilitating and, in extreme cases, incapacitating effects in patients.

Although by completely different mechanisms, the three adverse effects appear in the patient as fatigue, muscle weakness, and lack of strength, which finally reaches the point of limiting the mobility.

On one hand, the breakdown of proteins that form the striated musculature and the subsequent loss of muscle mass observed in myopathy due to steroids, cause a significant decrease in the ability of muscle to perform its work, appearing as muscle weakness and lack of strength.

On the other hand, the inhibition of insulin-stimulated glucose uptake or hyperglycemia by GCs, results in poor energy production in the muscle, which, as in the previous case, appears as muscle weakness and lack of strength.

Finally, the loss of bone mass caused by GCs also leads to a patient unable to perform muscular effort, due to the imminence of a fracture.

In relation to steroid myopathy, it is known that long-term use of GCs, at high doses, causes a significant decrease in striated muscle, due to direct catabolic effect on muscle tissue, which in turn is caused by means of the activation of the glucocorticoid receptor. This effect is perceived by the patient as weakness and fatigue, which limit or hinder the execution of daily activities, as basic as climbing or descending stairs or getting up from a chair.

Regarding hyperglycemia, it is known that GCs inhibit insulin-stimulated glucose uptake in muscle and adipose tissue, and block the suppressive effect of insulin on hepatic glucose production.

The net result of exposure to supra-physiological levels of GCs is an elevation of plasma glucose concentration and a compensatory increase in plasma insulin levels.

If the insulin increase is insufficient, diabetes can be developed or worsened if it actually existed. Patients presenting hyperglycemia induced by GCs generally have polyphagia, polydipsia, and polyuria, plus fatigue, weakness, and lack of concentration among other ailments.

In turn, it is known that muscle is not the only tissue of the musculoskeletal system affected by exposure to high physiological levels of GCs. Loss of bone mass is one of the main adverse effects of this group of drugs, mainly caused by a decreased bone formation and, to a lesser extent, increased bone resorption.

Glucocorticoids decrease the synthesis of 1.25-OH vitamin D and block its action; therefore, a deficit of calcium absorption through the gastrointestinal tract occurs. At the same time, there is an increase of calcium urinary excretion, causing a deficit of calcium available for bone mineralization. The net result of exposure to high levels of GCs is a considerable reduction in bone mass, which could lead to osteopenia or osteoporosis.

According to the above, one of the current approaches to the management of adverse effects associated with extended treatment with high doses of GCs, is gradual dose reduction, changing to another GC with lower incidence or severity of adverse effects, or even the discontinuation of GC treatment. This type of approach may compromise the clinical efficacy of treatment by not maintaining the required doses for optimal pharmacological effect.

Other approaches involve the use of physical exercises or drugs with pharmacological activity to counteract some specific adverse effects experienced by the patient. In other words, these are treatments to reduce the impact of specific adverse effects already present in patients treated with GCs, rather than preventing the occurrence of the same.

For instance, in the case of steroid myopathy, some authors suggest that aerobic exercise and physical fitness can reduce muscle weakness.

Although there are no definitive recommendations for treatment, it seems reasonable to limit patient immobilization and its consequences. Passive, active-assisted, and even active exercises, depending on the degree of weakness of each patient, help prevent muscle contractures and muscle atrophy. (Fernández C. M., et al. Miopatia esteroidea. Seminarios de la Fundación Española de Reunnatologia. 2008; Vol 9/No. 4:201-6).

In those conditions, various pharmacological alternatives for the treatment of muscle weakness associated with glucocorticoids have been proposed, such as potassium supplements (Dropcho E. J., Soong S. J. Steroid-induced weakness in patients with primary brain tumors. Neurology. 1991; 41:1235-9); nicotine (Laviano A., Meguid M. M., Guijarro A., Muscaritoli M., Cascino A., Preziosa I., et al. Antimyopathic effects of carnitine and nicotine. Curr Opin Clin Nutr Metab Care 2006; 9:442-8); and vitamin E and amino acids such as L-carnitine or creatine (Paddon-Jones D., Wolfe R. R., Ferrando A. A. Amino acid supplementation for reversing bed rest and steroid myopathies. J Nutr 2005; 135:S1809-12). None of these alternatives have shown a clear preventive or therapeutic effect on muscle weakness.

None of the approaches described in the state of the art provide a comprehensive and preventive treatment of adverse effects associated with prolonged use of high doses of GCs. The lack of such treatment leads to a suboptimal benefit of the pharmacological effects of GCs since, when an adverse effect occurs, it is necessary to reduce the dose, replace or even discontinue using the GCs to suppress the adverse effects.

The comprehensive approach to prevent or treat the adverse effects associated with extended use of high doses of GCs is important and necessary since, as mentioned above, GCs have the ability to exert effects on almost all systems; therefore, the adverse effects generated have no single etiology, but, on the contrary, result from countless interrelationships between different systems in the body, which ultimately are reflected in discomfort, and even in the risk to the integrity and life of the patient.

### SUMMARY OF THE INVENTION

The present invention is a solution to the problems identified above, as well as to other problems by providing a pharmaceutical preparation which prevents the adverse effects associated with long-term use of GCs. One exemplary preparation is a pharmaceutical composition comprising one or more amino acids; one or more vitamins; and one or more minerals; wherein the composition is formulated to prevent adverse effects associated with use of glucocorticoids.

In one preferred embodiment, the pharmaceutical composition comprises a first amino acid is present in an amount between 100 mg and 1,000 mg; a second amino acid is present in an amount between 50 mg and 500 mg; a third amino acid is present in an amount between 100 and 1,000 mg; a fourth amino acid is present in an amount 50 mg and 500 mg; a fifth amino acid is present in an amount between 1 g and 10 g; a first mineral is present in an amount between 25 mg and 1,000 mg; a second mineral is present in an amount between 50 μg and 500 μg; a third mineral is present in an amount between 200 mg and 2,500 mg; and first vitamin is present in an amount between 5 μg and 25 μg, equivalent to between 200 IU and 1,000 IU; and a second vitamin is present in an amount 25 μg and 300 μg.

The first object of the invention relates to a pharmaceutical formulation comprising mixtures of calcium, cholecalciferol, menaquinone-7, potassium, creatine, L-glutamine, L-valine, L-leucine, L-isoleucine, and chromium.

In addition, the invention discloses a method of preparing the pharmaceutical composition and its use as part of a treatment method that avoids the adverse effects associated with prolonged use of GCs.

The above described objects, as well as any objects which may apply, will be presented in detail and with the necessary sufficiency in the chapter below, which will constitute the foundation of claims.

### DESCRIPTION OF FIGURES.

FIG. 1 shows the method of preparing the preferred composition of the invention for a pilot batch of 20,000 capsules.

### DETAILED DESCRIPTION OF THE INVENTION

The invention summarized above and defined by the enumerated claims may be better understood by referring to the following description, which should be read in conjunction with the accompanying drawings. This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Prior to the detailed description of the invention, definitions of some terms will be described.

The term “comprising” should be understood as not limiting. For the expected purposes of the present invention, the term “containing” is considered preferred to the term “composed of”. If hereinafter a group is defined as comprising a number of embodiments, it is intended to encompass a group consisting of such embodiments preferably. The use of the letter “a” or the word “an” in an element of a claim does not limit the element to a single component, and may include the use of multiple numbers of the element.

In general terms, a “pharmaceutical dosage” should be understood as a formulation comprising an active ingredient in a specific amount in order to achieve a specific result.

The term “bioavailable”, as exposed in the present invention, refers to the measurement of the amount and speed with which an active ingredient contained in a pharmaceutical preparation reaches the general circulation and becomes available at the site of action.

The term IU, as understood by a person of ordinary skill in the art, refers to International Units, which is a measurement for the amount of some vitamins according to their biological activity.

As used in this application, the term “selected from a group consisting of” includes the selection of one or more members of the group cited. It does not require the election of only one member of the group unless stated otherwise.

The present invention discloses a novel pharmaceutical formulation for preventing the occurrence of adverse effects caused by prolonged use of GCs in different types of treatments. One exemplary preparation is a pharmaceutical composition comprising one or more amino acids; one or more vitamins; and one or more minerals; wherein the composition is formulated to prevent adverse effects associated with use of glucocorticoids.

This novel pharmaceutical dosage form to be used in the prevention of side effects associated with long-term treatment with GCs, when administered to a patient under treatment with high doses of these drugs, and for extended periods, prevents the onset of the three adverse effects that are considered critical due to their high frequency and potential debilitating effect to the patient. These effects are steroid myopathy, hyperglycemia and loss of bone mass.

It was found that executing a comprehensive pharmacological intervention for the simultaneous prevention of the three adverse effects due to GCs, caused a significant improvement in the quality of life of patients, expressed as an improvement in the feeling of weakness and lack of force. This is explained by the fact the three different etiologies that trigger this manifestation are under attack.

In order to counteract the said problems, the present invention discloses a pharmaceutical formulation containing a combination of vitamins, minerals, and amino acids, that when administered daily to a patient under prolonged treatment with high doses of GCs, avoids comprehensively the onset of the debilitating adverse effects most commonly seen during such treatments.

In this sense, the use of the formulation of the present invention allows the patient under treatment with GCs for lengthy periods, to maintain the necessary therapeutic doses in order to obtain the desired pharmacological effect, without the adverse effects which usually lead to discontinuation of treatment.

Types, salts, or forms of each of the product components were carefully selected to ensure they are absorbed and become bioavailable.

In one preferred embodiment, the pharmaceutical preparation contains one or more elements to avoid steroid myopathy, where the elements aimed at preventing steroid myopathy comprise one or more amino acids, including glutamine, valine, leucine, isoleucine, creatine, or other pharmaceutically acceptable salts of these amino acids. In one exemplary embodiment, the chemical form of glutamine is selected from the group consisting of L-glutamine, DL-glutamine, L-alanyl-L-glutamine, N-acetyl-L-glutamine, glutamic acid, alpha-ketoglutarate, or mixtures thereof. In yet a further embodiment, the chemical form of valine is selected from the group consisting of L-valine, DL-valine, or mixtures thereof. In another exemplary embodiment, the chemical form of leucine is selected from the group consisting of L-leucine, DL-leucine, or mixtures thereof. In addition, the chemical form of isoleucine is selected from the group consisting of L-isoleucine, DL-isoleucine, or mixtures thereof.

The amino acids are combined with potassium salts, which together favor building new muscle tissue and help the striated muscle to contract more efficiently and with greater strength.

One preferred embodiment, includes vitamins selected from the group consisting of vitamin D3 and vitamin K2. In a preferred embodiment the chemical form of vitamin D3 is selected from the group consisting of cholecalciferol, calciferol, ergocalciferol, or mixtures thereof. Similarly, in another preferred embodiment, the chemical form of vitamin K2 is selected from the group consisting of menaquinone-7, menaquinone-4, or mixtures thereof.

In addition to amino acids, one exemplary embodiment also contains minerals that are selected from the group consisting of potassium, calcium, and chromium, and salts thereof. In some embodiments, the chemical form of potassium is selected from the group consisting of potassium phosphate, potassium gluconate, potassium citrate, potassium acetate, potassium aspartate, potassium carbonate, potassium bicarbonate, potassium glycinate, potassium amino chelate, or mixtures thereof. In another embodiment, the chemical form of chromium is selected from the group consisting of chromium picolinate, chromium nicotinate, chromium polynicotinate, chromium chelate, chromium amino chelate, chromium GTF chelate, or mixtures thereof. In addition, the chemical form of calcium is selected from the group consisting of calcium carbonate, calcium citrate, calcium malate, calcium citrate malate, calcium ascorbate, calcium chelate or amino chelate, calcium gluconate, calcium glycinate, calcium aspartate, calcium succinate, calcium fumarate, calcium lactate gluconate or mixtures thereof.

In one further preferred embodiment, the therapeutic formulation used to prevent the occurrence of steroid myopathy is comprises the following components: L-glutamine, present in an amount which can vary between 100 mg and 1,000 mg, and more preferably between 200 and 500 mg; L-valine, present in an amount which can vary between 50 mg and 500 mg, and more preferably between 100 mg and 250 mg; L-leucine, present in an amount which can vary between 100 mg and 1,000 mg, and more preferably between 200 mg and 500 mg; Creatine, present in an amount which can vary between 1 g and 10 g, and more preferably between 2 g and 5 g; and potassium, present in an amount which can vary between 25 mg and 1,000 mg, and more preferably between 50 mg and 500 mg.

The pharmaceutical preparation also contains one or more elements intended to prevent hyperglycemia caused by GCs. In a preferred embodiment, the elements designed to prevent hyperglycemia due to GCs correspond to a combination of a chromium salt and the amino acid isoleucine, which together decrease the levels of blood glucose, specifically in hyperglycemia arising from a treatment with GCs. For example, a formulation for the therapeutic treatment to prevent the onset of hyperglycemia comprises L-isoleucine, present in an amount of between 50 mg and 500 mg, and more preferably between 100 mg and 300 mg; and Chromium as chromium picolinate, in a concentration between 50 μg and 600 μg, and more preferably between 100 μg and 300 μg.

The pharmaceutical preparation also contains one or more elements aimed at preventing loss of bone mass induced by GCs. In a preferred embodiment, the elements intended to prevent loss of bone mass by GCs correspond to one or more calcium salts, combined with cholecalciferol and menaquinone-7, which are cofactors necessary for this mineral to be absorbed and fixed in bone tissue. In one example, the formulation used in a therapeutic method to prevent loss of bone mass comprises calcium as calcium carbonate, in an amount of between 200 mg and 2,500 mg, and more preferably between 500 mg and 1,500 mg; cholecalciferol, in an amount of between 5 μg and 25 μg, equivalent to between 200 IU and 1,000 IU of vitamin D3, and more preferably between 10 μg and 20 μg, equivalent to between 400 IU and 800 IU of vitamin D3; and Vitamin K2 as menaquinone-7, in an amount of between 25 μg and 300 μg, and more preferably between 50 μg and 200 μg.

The formulation in accordance with one embodiment of the invention, is administered according to a number of available schemes, as selected by the treating physician. For example, the formulation can be administered as a single daily administration containing the total amount established for each component, separate from the administration of the glucocorticoid, for a period of at least two hours; or two daily administrations, every 12 hours, each containing 50% of the amount established for each component, separate from the administration of the glucocorticoid, for a period of at least two hours. It is contemplated that other treatment schemes can be used based on the physiology of the individual undergoing treatment.

The pharmaceutical preparation which is the object of the present invention is designed to be administered as a daily dose of one single stick pack or sachet, or four tablets or capsules, or combinations of tablets and capsules.

In a preferred embodiment of the invention, the pharmaceutical dosage includes a stick pack or sachet packaging containing the total daily dose plus a flavored base, as can be seen in the following Table.

TABLE 1
Pharmaceutical dosage including a stick pack or sachet
packaging containing the total daily dose

| COMPONENT | DAILY DOSE | AMOUNT PER UNIT |
|---|---|---|
| Creatine | Between 1 g and 10 g | Between 1 g and 10 g |
| L-glutamine | Between 100 mg and | Between 100 mg and |
| | 1,000 mg | 1,000 mg |
| L-valine | Between 50 mg and | Between 50 mg and |
| | 500 mg | 500 mg |
| L-leucine | Between 100 mg and | Between 100 mg and |
| | 1,000 mg | 1,000 mg |
| Potassium (as | Between 25 mg and | Between 25 mg and |
| potassium phosphate) | 1,000 mg | 1,000 mg |
| L-isoleucine | Between 50 mg and | Between 50 mg and |
| | 500 mg | 500 mg |
| Chromium (as | Between 50 μg and | Between 50 μg and |
| chromium picolinate) | 600 μg | 600 μg |
| Calcium (as | Between 200 mg and | Between 200 mg and |
| calcium carbonate) | 2,500 mg | 2,500 mg |
| Vitamin D3 (as | Between 200 IU and | Between 200 IU and |
| cholecalciferol) | 1,000 IU | 1,000 IU |
| Vitamin K2 (as | Between 25 μg and | Between 25 μg and |
| menaquinone-7) | 300 μg | 300 μg |

In another preferred form, the invention is designed to administer the daily required dose as multiple capsules or tablets containing each a fraction of the total daily dose; for example, four capsules or tablets containing each a quarter of the total required daily dose. The formulation is used in a therapeutic treatment to prevent the adverse effects of using GCs. The therapeutic treatment includes the administration, twice daily, of two capsules or tablets in the morning and two capsules or tablets in the evening, as it can be seen in the following Table. It is understood that the number of doses may be adjusted by the administering physician in accordance with the physiological conditions of the patient.

TABLE 2
Pharmaceutical dosage including four capsules or tablets
containing the total daily dose

| COMPONENT | DAILY DOSE | AMOUNT PER UNIT |
|---|---|---|
| L-glutamine | Between 100 mg and | Between 25 mg and |
| | 1,000 mg | 250 mg |
| L-valine | Between 50 mg and | Between 12.5 mg and |
| | 500 mg | 125 mg |
| L-leucine | Between 100 mg and | Between 25 mg and |
| | 1,000 mg | 250 mg |
| Potassium (as | Between 25 mg and | Between 6.25 mg and |
| potassium phosphate) | 1,000 mg | 250 mg |
| L-isoleucine | Between 50 mg and | Between 12.5 mg and |
| | 500 mg | 125 mg |
| Chromium (as | Between 50 μg and | Between 12.5 μg and |
| chromium picolinate) | 600 μg | 150 μg |
| Calcium (as | Between 200 mg and | Between 50 mg and |
| calcium carbonate) | 2,500 mg | 625 mg |
| Vitamin D3 (as | Between 200 IU and | Between 50 IU and |
| cholecalciferol) | 1,000 IU | 250 IU |
| Vitamin K2 (as | Between 25 μg and | Between 6.25 μg and |
| menaquinone-7) | 300 μg | 75 μg |

The preferred embodiment of the invention is a pharmaceutical dosage form containing an amount of L-glutamine (expressed as daily dose) which may vary between 100 mg and 1,000 mg, and preferably between 200 mg and 500 mg.

The preferred embodiment of the invention is a pharmaceutical dosage form containing an amount of L-valine (expressed as daily dose) which may vary between 50 mg and 500 mg, and preferably between 100 mg and 250 mg.

The preferred embodiment of the invention is a pharmaceutical dosage form containing an amount of L-leucine (as a daily dose) which may vary between 100 mg and 1,000 mg, and preferably between 200 mg and 500 mg.

The preferred embodiment of the invention is a pharmaceutical dosage form containing an amount of potassium (expressed as daily dose of elemental potassium) which may vary between 25 mg and 1,000 mg, and preferably between 50 mg and 500 mg.

The preferred embodiment of the invention is a pharmaceutical dosage form containing an amount of L-isoleucine (expressed as daily dose) that may vary between 50 mg and 500 mg, and preferably between 100 mg and 300 mg.

The preferred embodiment of the invention is a pharmaceutical composition containing an amount of chromium (expressed as daily dose of elemental chromium) that may vary between 50 μg and 600 μg, and preferably between 100 μg and 300 μg.

The preferred embodiment of the invention is a pharmaceutical composition containing an amount of calcium (expressed as daily dose of elemental calcium) that can vary from 200 mg to 2,500 mg, and preferably between 500 mg and 1,500 mg.

The preferred embodiment of the invention is a pharmaceutical composition containing an amount of cholecalciferol (expressed as daily dose) which can vary between 200 IU and 1,000 IU of vitamin D, and preferably between 400 IU and 800 IU of vitamin D.

The preferred embodiment of the invention is a pharmaceutical composition containing an amount of nnenaqionone-7 (expressed as daily dose) that can vary between 25 mg and 300 mg of vitamin K, and preferably between 50 mg and 200 mg of vitamin K2.

Further embodiments of the invention comprise the modifications listed below:

In one preferred embodiment of the invention, the preferred form of glutamine is L-glutamine, but DL-glutamine, L-alanyl-L-glutamine, N-acetyl-L-glutamine, glutamic acid, alpha-ketoglutarate or mixtures of the above can also be used.

In another preferred embodiment of the invention, the preferred form of valine is L-valine, but DL-valine or mixtures thereof can also be used.

In another preferred embodiment of the invention, the preferred form of leucine is L-leucine, but DL-leucine or mixtures thereof can also be used.

In another preferred embodiment of the invention, the preferred source of potassium is potassium phosphate, but potassium gluconate, potassium citrate, potassium acetate, potassium aspartate, potassium carbonate, potassium bicarbonate, potassium glycinate, potassium amino chelate, or mixtures thereof also can be used.

In another preferred embodiment of the invention, the preferred form of leucine is L-isoleucine, but DL-isoleucine or mixtures thereof can also be used.

In another preferred embodiment of the invention, the preferred source of chromium is chromium picolinate, but chromium nicotinate, chromium polynicotinate, chromium chelate, chromium amino chelate, chromium GTF chelate, or mixtures thereof can also be used.

In another preferred embodiment of the invention, the preferred source of calcium is calcium carbonate, but calcium citrate, calcium malate, calcium citrate malate, calcium ascorbate, calcium chelate or amino chelate, calcium gluconate, calcium glycinate, calcium aspartate, calcium succinate, calcium funnarate, calcium lactate gluconate or mixtures thereof can also be used.

In another preferred embodiment of the invention, the preferred form of vitamin D3 is cholecalciferol, but calciferol, ergocalciferol, or mixtures thereof can also be used.

In another preferred embodiment of the invention, the preferred form of vitamin K2 is menaquinone-7, but menaquinone-4 or mixtures thereof can also be used.

As described above, one preferred embodiment is a pharmaceutical composition, wherein a first amino acid is L-glutamine, present in an amount between 200 and 500 mg; a second amino acid is L-valine, present in an amount between 100 mg and 250 mg; a third amino acid is L-leucine, present in an amount between 200 mg and 500 mg; a fourth amino acid is L-isoleucine, present in an amount between 100 mg and 300 mg; a fifth amino acid is creatine, present in an amount between 2 g and 5 g; a first mineral is potassium, present in an amount between 50 mg and 500 mg; a second mineral is chromium, present in an amount between 100 μg and 300 μg; a third mineral is calcium, present in an amount between 500 mg and 1,500 mg; a first vitamin is D3 as cholecalciferol, present in an amount between 10 μg and 20 μg, equivalent to between 400 IU and 800 IU of vitamin D; and a second vitamin is K2 as menaquinone-7, present in an amount between 50 μg and 200 μg.

In a preferred embodiment, the pharmaceutical composition also includes an excipient. The excipient is selected from the group consisting of rice flour, colloidal silicon dioxide, and vegetable magnesium stearate.

### EXAMPLE 1

Solid Composition for a Batch of 20,000 Capsules

Three independent pilot batches of 20,000 capsules each, using the formulation described in the following table, were prepared:

TABLE 3
Pilot batch for 20,000 capsules using one of the
preferred compositions

| | AMOUNT PER | | AMOUNT PER | |
|---|---|---|---|---|
| COMPONENT | CAPSULE | | 20,000 CAPSULES | |
| L-glutamine | 62.5 | mg | 1,250.0 | g |
| L-valine | 37.5 | mg | 750.0 | g |
| L-leucine | 75.0 | mg | 1,500.0 | g |
| Dipotassium phosphate | 50.0 | mg | 1,000.0 | g |
| L-isoleucine | 37.5 | mg | 750.0 | g |
| Chromium picolinate | 0.4 | mg | 8.0 | g |
| Calcium carbonate | 625.0 | mg | 12,500.0 | g |
| Cholecalciferol | 5.0 | μg | 100.0 | mg |
| Menaquinone-7 | 25.0 | μg | 500.0 | mg |

Excipients for 20,000 capsules: rice flour, 1,837.1 g; colloidal silicon dioxide, 1,420.7 g; and vegetable magnesium stearate, 383.6 g. The capsule used was the standard “00” size, but other sizes can be utilized as recognized by a person of ordinary skill in the art.

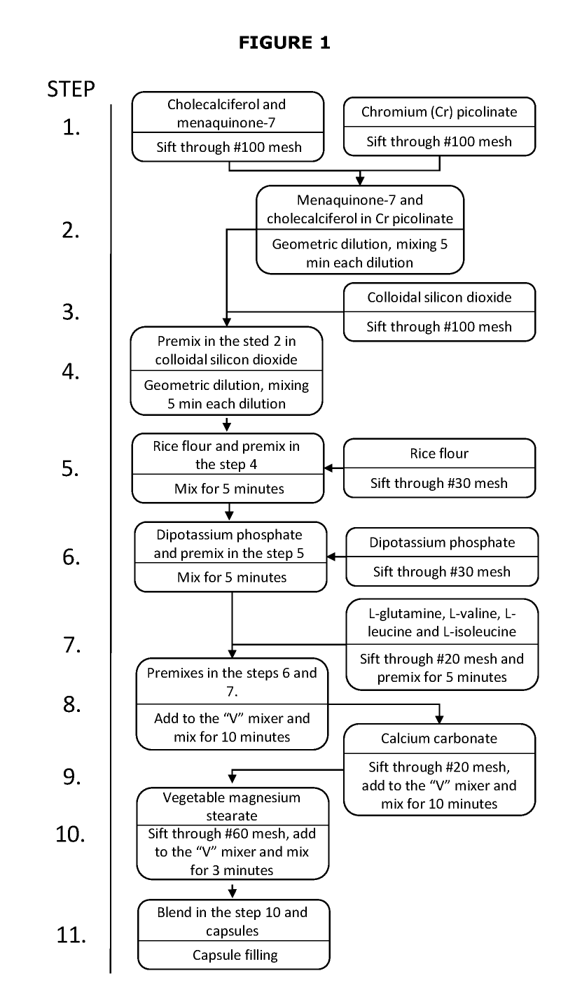

The preferred composition described in Table 3 was prepared according to the method described below and shown in FIG. 1.

a. Sifting cholecalciferol, menaquinone-7, and chromium picolinate through a mesh #100 or higher.

b. Performing a geometric dilution of cholecalciferol and menaquinone-7 using chromium picolinate as diluent, and mix for at least 3 minutes after each dilution. In some preferred embodiments the composition is mixed for at least 5 minutes.

c. Sifting colloidal silicon dioxide through a #60 mesh or higher. In a preferred embodiment a mesh #100 is used.

d. Performing a geometric dilution of the premix obtained in step 2 using colloidal silicon dioxide as diluent, and mix for at least 3 minutes after each dilution. In some preferred embodiments the composition is mixed for at least 5 minutes.

e. Sifting rice flour through a mesh #20 or higher (in some embodiments a #30 mesh is preferred), add to the premix obtained in step 4 and mix for at least 3 minutes. In some preferred embodiments the composition is mixed for at least 5 minutes.

f. Sifting dipotassium phosphate through a mesh #20 or higher (in some embodiments a #30 mesh is preferred), add to the premix obtained in step 5 and mix for at least 3 minutes. In some preferred embodiments the composition is mixed for at least 5 minutes.

g. Sifting L-glutamine, L-valine, L-leucine, and L-isoleucine through a mesh #20 or higher and mix for at least 3 minutes. In some preferred embodiments the composition is mixed for at least 5 minutes.

h. Adding premixes obtained in steps 6 and 7 in a “V” mixer and mix for at least 5 minutes. In some preferred embodiments the composition is mixed for at least 10 minutes.

i. Sifting calcium carbonate through a mesh #16 or higher (in one preferred embodiment a #20 mesh is used), add in the “V” mixer and mix for at least 5 minutes. In some preferred embodiments the composition is mixed for at least 10 minutes.

j. Sifting vegetable magnesium stearate through a #60 mesh or higher, add in the “V” mixer and mix for no more than 4 minutes. In some preferred embodiments the composition is mixed for at least 3 minutes.

k. Filling hard gelatin capsules to reach an average net weight of 1,070.0 mg per capsule. In some preferred embodiments a “#00” capsule is used.

In some preferred embodiments, the following components of the compositions may be utilized:

| | RANGE PER | RANGE PER |
|---|---|---|
| COMPONENT | CAPSULE | 20,000 CAPSULES |
| L-glutamine | Between 25 mg and | Between 500 g and |
| | 250 mg | 5,000 g |
| L-valine | Between 12.5 mg | Between 250 g and |
| | and 125 mg | 2,500 g |
| L-leucine | Between 25 mg and | Between 500 g and |
| | 250 mg | 5,000 g |
| Dipotassium | Between 6.25 mg | Between 125 g and |
| phosphate | and 250 mg | 5,000 g |
| L-isoleucine | Between 12.5 mg | Between 250 g and |
| | and 125 mg | 2,500 g |
| Chromium picolinate | Between 0.1 mg and | Between 2 g and 24 g |
| | 1.2 mg | |
| Calcium carbonate | Between 125 mg and | Between 2,500 g and |
| | 1,562.5 mg | 31,250 g |
| Cholecalciferol | Between 1.25 μg and | Between 25 mg and |
| | 6.25 μg | 125 mg |
| Menaquinone-7 | Between 6.25 μg and | Between 125 mg and |
| | 75 μg | 1,500 mg |

In yet other embodiments, the following components may be utilized:

| | RANGE PER | RANGE PER |
|---|---|---|
| COMPONENT | CAPSULE | 20,000 CAPSULES |
| L-glutamine | Between 50 mg and | Between 1,000 g and |
| | 125 mg | 2,500 g |
| L-valine | Between 25 mg and | Between 500 g and |
| | 62.5 mg | 1,250 g |
| L-leucine | Between 50 mg and | Between 1,000 g and |
| | 125 mg | 2,500 g |
| Dipotassium | Between 12.5 mg | Between 250 g and |
| phosphate | and 125 mg | 2,500 g |
| L-isoleucine | Between 25 mg and | Between 500 g and |
| | 75 mg | 1,500 g |
| Chromium picolinate | Between 0.2 mg and | Between 4 g and 12 g |
| | 0.6 mg | |
| Calcium carbonate | Between 312.5 mg | Between 6,250 g and |
| | and 937.5 mg | 18,750 g |
| Cholecalciferol | Between 2.5 μg and | Between 50 mg and |
| | 5 μg | 100 mg |
| Menaquinone-7 | Between 12.5 μg and | Between 250 mg and |
| | 50 μg | 1,000 mg |

Excipients for 20,000 capsules can be used and the following is one example of such excipient use: rice flour, between 1% and 10% of the total weight of the preparation; colloidal silicon dioxide, between 1% and 10% of the total weight of the preparation; and vegetable magnesium stearate, between 0.5% and 5.0% of the total weight of the preparation. The capsule used was the standard “00” size, but other sizes can be utilized as recognized by a person of ordinary skill in the art. As a result the concentration of components for each capsule would be the following:

| COMPONENT | RANGE PER CAPSULE (%) |
|---|---|
| L-glutamine | Between 3.5% and 8.5% |
| L-valine | Between 2.0% and 5.0% |
| L-leucine | Between 4.0% and 10.0% |
| Dipotassium phosphate | Between 2.5% and 6.5% |
| L-isoleucine | Between 2.0% and 5.0% |
| Chromium picolinate | Between 0.02% and 0.06% |
| Calcium carbonate | Between 35.0% and 85.0% |
| Cholecalciferol | Between 0.0003% and 0.0007% |
| Menaquinone-7 | Between 0.0014% and 0.0033% |
| Rice floor | Between 1.0% and 10.0% |
| Colloidal silicon dioxide | Between 1.0% and 10.0% |
| Vegetable magnesium | Between 0.5% and 5.0% |
| stearate | |

### EXAMPLE 2

Stability of the Composition

Using the three pilot batches as described in Example 1, stability studies under both storage conditions, accelerated and long-term, were carried out. The conditions of the studies were as follows:

Storage conditions for accelerated stability study: 40° C.±2° C./75%±5% of relative humidity.

Storage conditions for long-term stability study: 30° C.±2° C./65%±5% of relative humidity.

Duration of the accelerated stability study: 6 months

Duration of the long-term stability study: 36 months

Number of pilot batches used in the studies: 3

Bottling and packaging material: 250 mL white HDPE bottle, ribbed 45 mm CRC and heat sealed, plus cotton, silica desiccant, and folding carton.

The physical parameters evaluated in both types of studies were the following: physical description, moisture content, average weight, weight variation, disintegration time.

Chemical parameters evaluated in both types of studies were the following: assay of vitamin D as cholecalciferol, assay of calcium as calcium carbonate, assay of chromium as chromium picolinate, and dissolution of calcium as calcium carbonate.

Microbiological parameters evaluated in both types of studies were the following: total aerobic microbial count (TAMC), total combined yeasts and molds count (TYMC), verification of the absence of E. coli, Salmonella, S. aureus, and Pseudomonas spp.

The studies were conducted in accordance with ICH Q1A (R2) Guidelines and the Pharmacopoeia of the United States of America, version 38, chapters <2091>, <2040>, and Dietary Supplements—Vitamins.

The results of both studies successfully met the acceptance criteria established by both the Pharmacopoeia of the United States of America and ICH Q1A (R2) Guidelines.

1. A pharmaceutical composition, comprising:
an amino acid, selected from the group consisting of glutamine, valine, leucine, isoleucine, creatine; pharmaceutically acceptable salts thereof, and mixtures thereof;
a vitamin selected from the group consisting of vitamin D3 and vitamin K2, and mixtures thereof;
and a mineral selected from the group consisting of potassium, calcium, and chromium, salts thereof, and mixtures thereof.

2. (canceled)

3. The pharmaceutical composition of claim 1, wherein the glutamine is selected from the group consisting of L-glutamine, DL-glutamine, L-alanyl-L-glutamine, N-acetyl-L-glutamine, glutamic acid, alpha-ketoglutarate, or mixtures thereof.

4. The pharmaceutical composition of claim 1, wherein the valine is selected from the group consisting of L-valine, DL-valine, or mixtures thereof.

5. The pharmaceutical composition of claim 1, wherein the Leucine is selected from the group consisting of L-leucine, DL-leucine, or mixtures thereof.

6. The pharmaceutical composition of claim 1, wherein the of isoleucine is selected from the group consisting of L-isoleucine, DL-isoleucine, or mixtures thereof.

7. (canceled)

8. The pharmaceutical composition of claim 1, wherein the vitamin D3 is selected from the group consisting of cholecalciferol, calciferol, ergocalciferol, or mixtures thereof.

9. The pharmaceutical composition of claim 1, wherein the vitamin K2 is selected from the group consisting of menaquinone-7, menaquinone-4, or mixtures thereof.

10. (canceled)

11. The pharmaceutical composition of claim 1, wherein the potassium is selected from the group consisting of potassium phosphate, potassium gluconate, potassium citrate, potassium acetate, potassium aspartate, potassium carbonate, potassium bicarbonate, potassium glycinate, potassium amino chelate, or mixtures thereof.

12. The pharmaceutical composition of claim 1, wherein the chromium is selected from the group consisting of chromium picolinate, chromium nicotinate, chromium polynicotinate, chromium chelate, chromium amino chelate, chromium GTF chelate, or mixtures thereof.

13. The pharmaceutical composition of claim 1, wherein the calcium is selected from the group consisting of calcium carbonate, calcium citrate, calcium malate, calcium citrate malate, calcium ascorbate, calcium chelate or amino chelate, calcium gluconate, calcium glycinate, calcium aspartate, calcium succinate, calcium fumarate, calcium lactate gluconate or mixtures thereof.

14. The pharmaceutical composition of claim 1, wherein:
a first amino acid is present in an amount between 100 mg and 1,000 mg;
a second amino acid is present in an amount between 50 mg and 500 mg;
a third amino acid is present in an amount between 100 and 1,000 mg;
a fourth amino acid is present in an amount between 50 mg and 500 mg;
a fifth amino acid is present in an amount between 1 g and 10 g;
a first mineral is present in an amount between 25 mg and 1,000 mg;
a second mineral is present in an amount between 50 μg and 600 μg;
a third mineral is present in an amount between 200 mg and 2,500 mg;
a first vitamin is present in an amount between 5 μg and 25 μg, equivalent to between 200 IU and 1,000 IU; and
a second vitamin is present in an amount between 25 μg and 300 μg.

15. The pharmaceutical composition of claim 14, wherein:
The first amino acid is L-glutamine, present in an amount between 200 and 500 mg;
The second amino acid is L-valine, present in an amount between 100 mg and 250 mg;
The third amino acid is L-leucine, present in an amount between 200 mg and 500 mg;
The fourth amino acid is L-isoleucine, present in an amount between 100 mg and 300 mg;
The fifth amino acid is creatine, present in an amount between 2 g and 5 g;
The first mineral is potassium, present in an amount between 50 mg and 500 mg;
The second mineral is chromium, present in an amount between 100 μg and 300 μg;
The third mineral is calcium, present in an amount between 500 mg and 1,500 mg;
The first vitamin is D3 as cholecalciferol, present in an amount between 10 μg and 20 μg, equivalent to between 400 IU and 800 IU of vitamin D; and
The second vitamin is K2 as menaquinone-7, present in an amount between 50 μg and 200 μg.

16. The pharmaceutical composition of claim 1; further comprising an excipient.

17. The pharmaceutical composition of claim 16, wherein the excipient is selected from the group consisting of rice flour, colloidal silicon dioxide, and vegetable magnesium stearate.

18. The pharmaceutical composition of claim 1, used in a therapeutic treatment.

19. A method of preparing a pharmaceutical composition that prevents adverse effects associated with the use of glucocorticoids, comprising:
sifting cholecalciferol, menaquinone-7, and chromium picolinate through a mesh #60 or higher;
geometrically diluting cholecalciferol and menaquinone-7 using chromium picolinate as diluent, and mix for at least 3 minutes after each dilution to obtain a geometrically diluted pre-mix;
sifting colloidal silicon dioxide through a mesh #60 or higher;
geometrically diluting the obtained pre-mix using colloidal silicon dioxide as diluent, and mix for at least 3 minutes after each dilution;
sifting rice flour through a mesh #20 or higher, add to the premix and mix for at least 3 minutes;
sifting dipotassium phosphate through a mesh #20 or higher, add to the premix and mix for at least 3 minutes;
sifting L-glutamine, L-valine, L-leucine, and L-isoleucine through a mesh #20 or higher and mix for at least 3 minutes to obtain a second pre-mix;
combining the pre-mix and the second pre-mix in a “V” mixer and mix for at least 5 minutes;
sifting calcium carbonate through a mesh #16 or higher, add in the “V” mixer and mix for at least 5 minutes;
sifting vegetable magnesium stearate through a mesh #60 or higher, add in the “V” mixer and mix for not more than 4 minutes; and
filling hard gelatin capsules.

20. A treatment method to prevent the adverse effects associated with the use of glucocorticoids, which comprises oral administration of the pharmaceutical composition of claim 1.
